(19) United States
(12) Patent Application Publication
(10) Pub. No.: US 2023/0263753 A1
(43) Pub. Date: Aug. 24, 2023

(54) SOFT CAPSULE COMPRISING B-HYDROXYBUTYRIC ACID

(71) Applicant: NANJING NUTRABUILDING BIO-TECH CO., LTD., Nanjing (CN)

(72) Inventors: Long JIANG, Lewisville, TX (US); Xuyang SUN, Nanjing (CN); Ronghua YI, Nanjing (CN); Shawn WELLS, Nanjing (CN); Kylin LIAO, Nanjing (CN)

(21) Appl. No.: 18/172,020

(22) Filed: Feb. 21, 2023

(30) Foreign Application Priority Data
Mar. 21, 2022 (CN) 18172020

(51) Int. Cl. A61K 31/19; A61K 9/48

(52) U.S. Cl. CPC A61K 31/19; A61K 9/4825; A61K 9/4833; A61K 9/4816; A61K 9/4858; A61K 9/4866

(57) ABSTRACT

Among others, this invention provides a soft capsule comprising β-hydroxybutyric acid (BHB) or a pharmaceutically acceptable salt, ester, liposome, or mixture thereof as active ingredient, as well as method of making such soft capsule.

### CROSS REFERENCE TO RELATED APPLICATION

This application claims the benefit of a PCT International Application Number PCT/CN2022/077108, filed on Feb. 21, 2022, the content of which is hereby incorporated by reference in its entirety for all purposes.

### FIELD OF THE INVENTION

This invention relates to a soft capsule comprising β-hydroxybutyric acid (BHB) or a pharmaceutically acceptable salt, ester, liposome, or mixture thereof as active ingredient, as well as method of making such soft capsule.

### BACKGROUND OF THE INVENTION

Ketone bodies are small, lipid-derived molecules that provide energy to tissues when the supply of glucose is too low for the body's energetic needs, such as during periods of prolonged exercise, starvation, or absence of dietary carbohydrates. During fasting, after muscle and liver stores of glycogen are depleted, fatty acids are mobilized from adipocytes and transported to the liver for conversion to ketone bodies. Ketone bodies are then distributed via blood circulation to metabolically active tissues, such as muscle or brain, where they are metabolized into acetyl-CoA and eventually ATP. The term ketone bodies usually include three molecules that are generated during ketogenesis: β-hydroxybutyrate (also known as μ-Hydroxybutyric Acid, 3-Hydroxybutyrate, or BHB), acetoacetate, and acetone. Most of the dynamic range in ketone body levels is in the form of BHB. When ketogenesis is activated, such as during fasting, blood levels of BHB rise much faster than either acetoacetate or acetone. As such, β-hydroxybutyrate (BHB) represents an essential carrier of energy from the liver to peripheral tissues when the supply of glucose is too low for the body's energetic needs. In addition to its activity as an energetic metabolite, BHB is increasingly understood to have cellular signaling functions. These signaling functions of BHB broadly link the outside environment to epigenetic gene regulation and cellular function, and their actions may be relevant to a variety of human diseases as well as human aging (Annu Rev Nutr. 2017 Aug. 21; 37: 51-76.doi: 10.1146/annurev-nutr-071816-064916).

Owing to the reported benefits, BHB was formulated as a pharmaceutical composition or dietary supplement for health benefits. These exogenous BHB has been widely sold commercially for quick entry and maintenance of ketogenic status, and are usually taken in the form of BHB salts or esters.

Soft capsules are a single-unit solid dosage form, consisting of a liquid or semi-solid fill enveloped by a one-piece hermetically sealed outer shell. Traditionally, gelatin is used almost exclusively as shell-forming material of soft capsules. Recent years, synthetic and/or plant-derived materials have been described as shell-forming material. Thus, depending on the polymer forming the shell, soft capsules can be divided into two categories, namely soft gelatin capsules or soft-gels, and non-gelatin soft capsules.

Soft capsules are modern and effective pharmaceutical dosage form for administration of many medicinal products and food supplements formulations. The fill may be selected from any of a wide variety of substances that are compatible with the shell. Soft capsule formulations have recently become of greater interest in the formulation of products for oral administration, since soft capsules can improve bioavailability of the active ingredient capsulated within the shell, reduce risk of oxidation and degradation, reduce discomfort of the gastrointestinal tract, and permit accurate delivery of a unit dose. In addition, soft capsules provide a dosage form for drugs with low aqueous solubility. And this dosage form is easy to swallow and need not be flavored, in fact, it can mask the undesirable taste of the fill materials.

In view of the state of the art, there is no published studies about the soft capsule formulation comprising BHB or derivatives and methods of making and administering such dosage forms. It would be of value to have a soft capsule formulation comprising BHB, which can mask the undesirable taste of BHB, such as the sour taste of BHB acid, the astringent taste of BHB salts, and the odor of BHB ester, etc.

### SUMMARY OF THE INVENTION

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

The invention is directed to soft capsule compositions comprising β-Hydroxybutyric Acid (BHB), or physiologically acceptable BHB salt, BHB ester, BHB liposome, or a mixture thereof, which exhibit excellent bioavailability when orally ingested, stability when stored for prolonged period of times, and mask the unpleasant taste of BHB acid, BHB salt, BHB ester, BHB liposome, or a mixture thereof.

In one aspect, the invention provides a soft capsule comprising an outer shell and a fill material within the outer shell, wherein the outer shell comprises a gelatin or a plant-based material; the fill material comprises an active ingredient which is β-hydroxybutyric acid (BHB), a pharmaceutically acceptable salt, ester, liposome, or a mixture thereof. In some embodiments, the BHB can be in D-form, L-form or a mixture of D- and L-forms.

In some embodiments, the outer shell further comprises a humectant and water, the humectant is selected from polyethylene glycol 200, polyethylene glycol 400, polyethylene glycol 600, polyethylene glycol 800, polyethylene glycol 1000, polyethylene glycol 1500, polyethylene glycol 2000, glycerin, sorbitol, polydextrose, maltitol, or a mixture thereof.

Humectants can be used to suppress the water activity of the soft capsule. In some embodiments, the humectant is selected from glycerin, sorbitol or polyethylene glycol 400.

In some embodiments, the active ingredient has a mass percentage ranging from 20% to 80% of the total weight of the capsule; the gelatin or plant-based material has a mass percentage ranging from 10% to 40% of the total weight of the capsule; the humectant has a mass percentage ranging from 2% to 20% of the total weight of the capsule; the water has a mass percentage ranging from 2% to 20% of the total weight of the capsule. In some embodiments, the active ingredient may have a mass percentage ranging from 1% to 99%, 50% to 85%, 20% to 80%, preferably 25% to 75%, more preferably 30% to 70% of the total weight of the capsule; the gelatin or plant-based material may have a mass percentage ranging from 1% to 40%, 10% to 40%, 10% to 30%, preferably 15% to 30%, more preferably 18% to 26% of the total weight of the capsule; the humectant may have a mass percentage ranging from 0.5% to 40%, 2% to 20%, 5% to 30%, preferably 3% to 18%, more preferably 4% to 15% of the total weight of the capsule; the water may have a mass percentage ranging from 0.1% to 20%, 2% to 20%, 5% to 15%, preferably 3% to 15%, more preferably 4% to 10% of the total weight of the capsule.

In some embodiments, the gelatin has a bloom strength of 100-300; the plant-based material/alternative is starch or colloid. In some embodiments, the gelatin is made from bones, skins, muscles, tendons, scales and other connective tissue rich in collagen from pigs, cows, chickens, fish, or beef. In some embodiments, the gelatin has a bloom strength of 50-350, preferably 100-300, more preferably 150-300.

In some embodiments, the starch is selected from oxidized starch, starch dispersion, starch derivatives, starch hydrolysate, wet heat-treated starch, acid-treated starch, or a mixture thereof the colloid is selected from carrageenan, mannan gum, xanthan gum, alginate, or a mixture thereof. In some embodiments, carrageenan is κ-carrageenan, ι-carrageenan, or λ-carrageenan; mannan gum is locust bean gum, konjac gum, or guar gum; alginate is sodium alginate.

In some embodiments, the pharmaceutically acceptable salt, ester is selected from BHB potassium salt, BHB sodium salt, BHB calcium salt, BHB magnesium salt, BHB calcium magnesium salt, BHB triglyceride. In some embodiments, the pharmaceutically acceptable salt, ester is selected from BHB sodium salt, BHB calcium magnesium salt, BHB triglyceride.

In some embodiments, the pharmaceutically acceptable salt, ester is selected from BHB sodium salt, BHB calcium magnesium salt, BHB triglyceride.

In some embodiments, the fill material further comprises a suspending agent, the suspending agent is selected from polyethylene glycol 200, polyethylene glycol 400, polyethylene glycol 600, polyethylene glycol 800, polyethylene glycol 1000, polyethylene glycol 1500, polyethylene glycol 2000, glycerin, sorbitol, polydextrose, maltitol and a mixture thereof.

In some embodiments, the suspending agent is selected from glycerin, polyethylene glycol 400 or sorbitol. In some embodiments, when the active ingredient in the fill material is BHB acid or BHB salt, the fill material further comprises a suspending agent.

In some embodiments, the suspending agent has a mass percentage ranging from 3% to 45% of the total weight of the capsule. The mass percentage of the suspending agent may be 1% to 99%, 15% to 50%, 3% to 45%, preferably 4% to 40%, more preferably 5% [symbol] 35%.

In some embodiments, the fill material is in the form of solution, suspension, or paste.

In some embodiments, the fill material is in the form of suspension. In some embodiments, the fill material is in the form of suspension by mixing the active ingredient with the suspending agent.

In some embodiments, the weight of the fill material ranges from 80 to 2500 mg, and the total weight of the soft capsule ranges from 120 to 3750 mg. The weight of the fill material may be 80 to 2500 mg, 100 to 2500 mg, preferably 90 to 2250 mg, more preferably 100 to 2000 mg; the total weight of the soft capsule may be 120 to 3750 mg, 150 to 3750 mg, preferably 130 to 3500 mg, more preferably 140 to 3000 mg.

In some embodiments, the soft capsule is used as nutritional supplement or pharmaceutical composition.

In another aspect, the invention provides a method for preparing the soft capsule as described above, comprising the steps of (a) preparing β-hydroxybutyric acid (BHB), or a pharmaceutically acceptable salt, ester, liposome or mixture thereof, to form a fill composition; (b) preparing and formulating an outer shell using gelatin or a plant-based material; and (c) encapsulating the fill material into the outer shell.

In some embodiments, the method further comprising a step of shaping, drying, quality inspection, packaging, or storage.

In some embodiments, step (a) comprises mixing the BHB or pharmaceutically acceptable salt with a suspending agent together, heating the mixture to a temperature of 35-60° C., preferably 45-50° C., and stirring, to form a homogenous mixture; or heating the pharmaceutically acceptable ester or liposome to a temperature of 35-60° C., preferably 45-50° C., and stirring. In some embodiments, the stirring is preferably performed under high shear conditions.

In some embodiments, step (b) comprises mixing the gelatin or plant-based material with a humectant and water together, stirring and heated to 50-100° C., preferably 60-95° C., de-aerating and filtering, to formulate an outer shell liquid. In some embodiments, when the outer shell comprises gelatin, the heating temperature of step (b) is 50-80° C., preferably 60-70° C.; when the outer shell comprises plant-based material, the heating temperature of step (b) is 80-100° C., preferably 90-95° C.

In yet another aspect, the invention relates to a method for providing health benefits in a subject, comprising administering the soft capsule composition comprising β-Hydroxybutyric acid (BHB), or pharmaceutically acceptable salt, ester, liposome, or a mixture thereof according to this invention.

Examples of the health benefits includes but limited to supporting weight loss, controlling appetite, offering longer-lasting energy, amplifying concentration level and mental clarity, regulating blood sugar and insulin levels, or burning off body's fat.

In some embodiments, the subject is a mammal, preferably, a human.

The soft capsule according to this invention can mask the undesirable taste of BHB, such as the sour taste of BHB acid and BHB liposome, the astringent taste of BHB salt, and/or the odor taste of BHB ester, and the like, so it need not be flavored; and it is easy to swallow, can improve bioavailability of the active ingredient, reduce risk of oxidation and degradation, reduce discomfort of the gastrointestinal tract, improve storage stability, and permit accurate delivery of a unit dose.

### DETAILED DESCRIPTION OF THE INVENTION

Reference will now be made in detail to the preferred embodiments of the invention, examples of which are further illustrated. While the invention will be described in conjunction with the preferred embodiments, it will be understood that they are not intended to limit the invention to these embodiments. To the contrary, the invention is intended to cover alternatives, modifications and equivalents, which may be included within the spirit and scope of the invention as defined by the claims. Furthermore, in the detailed description of the present invention, numerous specific details are set forth in order to provide a thorough understanding of the present invention. However, it will be obvious to one of ordinary skill in the art that the present invention may be practiced without these specific details. In other instances, well known methods, procedures, components, and other features have not been described in detail as not to unnecessarily obscure aspects of the present invention.

### Definitions

As used herein, the term “or” is meant to include both “and” and “or”. In other words, the term “or” may also be replaced with “and/or.”

As used herein, the singular forms “a,” “an” and “the” are intended to include the plural forms as well, unless the context clearly indicates otherwise.

As used herein, the term “subject” or “patient” is used interchangeably and as used herein mean any mammal including but not limited to human beings including a human patient or subject to which the compositions of the invention can be administered. The term “mammal” includes human patients and non-human primates, as well as experimental animals such as rabbits, rats, and mice, and other animals.

The term “unit dose” refers to a dosage form that is configured to deliver a specified quantity or dose of composition or component thereof.

As used herein, the term “comprise” or “include” and their conjugations, refer to a situation wherein said terms are used in their non-limiting sense to mean that items following the word are included, but items not specifically mentioned are not excluded. It also encompasses the more limiting verb ‘to consist essentially of’ and ‘to consist of’.

As used herein, the term “pharmaceutically acceptable” means pharmaceutically, physiologically, alimentarily, and/or nutritionally acceptable, and refers to those compositions or combinations of agents, materials, or compositions, and/or their dosage forms, which are within the scope of sound medical judgment, suitable for use in contact with the tissues of human beings and animals without excessive toxicity, irritation, allergic response, or other problem or complication, commensurate with a reasonable benefit/risk ratio.

Chemical structure of β-hydroxybutyrate, also known as 3-hydroxybutyrate, β-HB, BHB, or beta-hydroxybutyrate, is shown below.

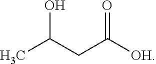

BHB is a chiral molecule at the 3′ hydroxyl group, and there are two enantiomers, R/D and S/L.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for.

### EXAMPLES

The following example illustrates the preparation of soft capsule of the present subject matter.

### Example 1

This Example discloses a soft capsule formulation comprising BHB acid. The formulation is shown in the Table below.

| | | |
|---|---|---|
| Component name | Mass (kg) | Mass percentage (%) |

Filling material:

| | | |
|---|---|---|
| BHB acid | 2.50 | 33.33 |
| Glycerin | 2.50 | 33.33 |

Outer shell:

| | | |
|---|---|---|
| Gelatin | 1.47 | 19.60 |
| Glycerin | 0.60 | 8.00 |
| Water | 0.43 | 5.73 |

The formulation was prepared as follows:

### Step 1: Fill Material Preparation

2.5 kg BHB acid and 2.5 kg glycerin were mixed and heated to 45° C. Then the mixture was stirred at 22000 rpm for 5 min under high shear conditions until homogenous mixture was obtained.

### Step 2: Molten Gel Preparation

In a separate container, 1.47 kg gelatin, 0.60 kg glycerin, and 1.47 kg water were mixed together, and stirred at 70° C. for 3 h until gelatin melts. Then, the molten gel was de-aerated under vacuum, and filtered using an 80-mesh screen. The gel mass was kept in a storage tank at constant temperature of 70° C. until used for encapsulation. During the gel manufacturing process, the ambient temperature is 20° C. and the relative humidity is 45%.

### Step 3: Encapsulation

The soft capsule is prepared by encapsulating fill material in a gel shell using a rotary die encapsulation process. In the encapsulation process, two gel ribbons are manufactured and pass between a twin set of rotating dies. As the ribbons meet, the filling formulation to be encapsulated is precisely injected between them. The capsule halves are sealed and ejected by the continuous rotation of the dies. During the encapsulation process, the ambient temperature is 20° C. and the relative humidity is 45%. The content weight is 100 mg.

### Step 4: Drying

An intelligent soft capsule tumbler dryer was used for shaping, drying and polishing. During the process, the ambient temperature is 20° C. and the relative humidity is 30%. After drying, the moisture content of the shell is 5.73%.

### Step 5: Quality Inspection

After drying, the soft capsules were placed on the lamp inspection platform for quality inspection. During the process, the ambient temperature is 20° C. and the relative humidity is 45%.

### Step 6: Packaging

During the process, the ambient temperature is 20° C. and the relative humidity is 30%. Qualified soft capsules were packed.

### Step 7: Storage

During the process, the ambient temperature is 20° C. and the relative humidity is 45%. The finished product of BHB soft capsules were packed and stored. The soft capsule is spherical, the weight of filling material (content weight) is 100 mg, and total weight of the soft capsule is about 140 mg.

### Example 2

This Example discloses a soft capsule formulation comprising BHB ester. The formulation is shown in the Table below.

| | | |
|---|---|---|
| Component name | Mass (kg) | Mass percentage (%) |

Filling material:

| | | |
|---|---|---|
| BHB ester | 10.00 | 55.56 |

Outer shell:

| | | |
|---|---|---|
| Gelatin | 4.40 | 24.44 |
| Sorbitol | 2.00 | 11.11 |
| Water | 1.60 | 8.89 |

The formulation was prepared as follows:

### Step 1: Fill Material Preparation

10 kg of BHB ester, being a liquid, was heated to 45° C. Then the liquid was stirred at 20000 rpm for 5min under high shear conditions.

### Step 2: Molten Gel Preparation

In a separate container, 4.40 kg gelatin, 2.00 kg sorbitol, and 5.28 kg water were mixed together, and stirred at 60° C. for 4h until gelatin melts. Then, the molten gel was de-aerated under vacuum, and filtered using an 80-mesh screen. The gel mass was then kept in a storage tank at constant temperature of 60° C. until used for encapsulation. During the gel manufacturing process, the ambient temperature is 20° C. and the relative humidity is 40%.

### Step 3: Encapsulation

The soft capsule is prepared by encapsulating fill material in a gel shell using a rotary die encapsulation process. In the encapsulation process, two gel ribbons are manufactured and pass between a twin set of rotating dies. As the ribbons meet, the filling formulation to be encapsulated is precisely injected between them. The capsule halves are sealed and ejected by the continuous rotation of the dies. During the encapsulation process, the ambient temperature is 20° C. and the relative humidity is 40%. The content weight is 250 mg.

### Step 4: Drying

An intelligent soft capsule tumbler dryer was used for shaping, drying and polishing. During the process, the ambient temperature is 20° C. and the relative humidity is 30%. After drying, the moisture content of the shell is 8.89%.

### Step 5: Quality Inspection

After drying, the soft capsules were placed on the lamp inspection platform for quality inspection. During the process, the ambient temperature is 20° C. and the relative humidity is 40%.

### Step 6: Packaging

During the process, the ambient temperature is 20° C. and the relative humidity is 30%. Qualified soft capsules were packed.

### Step 7: Storage

During the process, the ambient temperature is 20° C. and the relative humidity is 40%. The finished product of BHB soft capsules were packed and stored. The soft capsule is spherical, the weight of filling material (content weight) is 250 mg, and total weight of the soft capsule is about 450 mg.

### Example 3

This Example discloses a soft capsule formulation comprising BHB sodium salt. The formulation is shown in the Table below.

| | | |
|---|---|---|
| Component name | Mass (kg) | Mass percentage (%) |

Filling material:

| | | |
|---|---|---|
| BHB sodium salt | 15.00 | 48.39 |
| Polyethylene glycol 400 | 5.00 | 16.13 |

Outer shell:

| | | |
|---|---|---|
| Gelatin | 6.60 | 21.29 |
| Polyethylene glycol 400 | 1.65 | 5.32 |
| Water | 2.75 | 8.87 |

The formulation was prepared as follows:

### Step 1: Fill Material Preparation

15 kg BHB sodium salt and 5 kg polyethylene glycol 400 were mixed together and heated to 45° C. Then the mixture was stirred at 18000 rpm for 7 min under high shear conditions until homogenous mixture was obtained.

### Step 2: Molten Gel Preparation

In a separate container, 6.60 kg gelatin, 1.65 kg polyethylene glycol 400, and 7.92 kg water were mixed together, and stirred at 65° C. for 3.5 h until gelatin melts. Then, the molten gel was de-aerated under vacuum, and filtered using an 80-mesh screen, and then kept in a storage tank at constant temperature of 65° C. until used for encapsulation. During the gel manufacturing process, the ambient temperature is 22° C. and the relative humidity is 35%.

### Step 3: Encapsulation

The soft capsule is prepared by encapsulating fill material in a gel shell using a rotary die encapsulation process. In the encapsulation process, two gel ribbons are manufactured and pass between a twin set of rotating dies. As the ribbons meet, the filling formulation to be encapsulated is precisely injected between them. The capsule halves are sealed and ejected by the continuous rotation of the dies. During the encapsulation process, the ambient temperature is 22° C. and the relative humidity is 20%. The content weight is 500 mg.

### Step 4: Drying

An intelligent soft capsule tumbler dryer was used for shaping, drying and polishing. During the process, the ambient temperature is 22° C. and the relative humidity is 20%. After drying, the moisture content of the shell is 8.87%.

### Step 5: Quality Inspection

After drying, the soft capsules were placed on the lamp inspection platform for quality inspection. During the process, the ambient temperature is 22° C. and the relative humidity is 35%.

### Step 6: Packaging

During the process, the ambient temperature is 22° C. and the relative humidity is 20%. Qualified soft capsules were packed.

### Step 7: Storage

During the process, the ambient temperature is 22° C. and the relative humidity is 35%. The finished product of BHB soft capsules were packed and stored. The soft capsule is spherical, the weight of filling material (content weight) is 500 mg, and the total weight of soft capsule is about 775 mg.

### Example 4

This Example discloses a soft capsule formulation comprising BHB liposome. The formulation is shown in the Table below.

| | | |
|---|---|---|
| Component name | Mass (kg) | Mass percentage (%) |

Filling material:

| | | |
|---|---|---|
| BHB liposome | 20.00 | 66.67 |

Outer shell:

| | | |
|---|---|---|
| Gelatin | 5.90 | 19.67 |
| Glycerin | 2.40 | 8.00 |
| Water | 1.70 | 5.66 |

### Step 1: Fill Material Preparation

20 kg of BHB liposome, being a liquid, was heated to 50° C. Then the liquid was stirred at 22000 rpm for 5min under high shear conditions.

### Step 2: Molten Gel Preparation

In a separate container, 5.90 kg gelatin, 2.40 kg glycerin, and 5.90 kg water were mixed together, and stirred at 60° C. for 4 h until gelatin melts. Then, the molten gel was de-aerated under vacuum, and filtered using an 80-mesh screen, and then kept in a storage tank at constant temperature of 60° C. until used for encapsulation. During the gel manufacturing process, the ambient temperature is 20° C. and the relative humidity is 45%.

### Step 3: Encapsulation

The soft capsule is prepared by encapsulating fill material in a gel shell using a rotary die encapsulation process. In the encapsulation process, two gel ribbons are manufactured and pass between a twin set of rotating dies. As the ribbons meet, the filling formulation to be encapsulated is precisely injected between them. The capsule halves are sealed and ejected by the continuous rotation of the dies. During the encapsulation process, the ambient temperature is 20° C. and the relative humidity is 45%. The content weight is 1000 mg.

### Step 4: Drying

An intelligent soft capsule tumbler dryer was used for shaping, drying and polishing. During the process, the ambient temperature is 20° C. and the relative humidity is 30%. After drying, the moisture content of the shell is 5.66%.

### Step 5: Quality Inspection

After drying, the soft capsules were placed on the lamp inspection platform for quality inspection. During the process, the ambient temperature is 20° C. and the relative humidity is 30%.

### Step 6: Packaging

During the process, the ambient temperature is 20° C. and the relative humidity is 30%. Qualified soft capsules were packed.

### Step 7: Storage

During the process, the ambient temperature is 20° C. and the relative humidity is 45%. The finished product of BHB soft capsules were packed and stored. The soft capsule is spherical, the weight of filling material (content weight) is 1000 mg, and the total weight of soft capsule is about 1500 mg.

### Example 5

This Example discloses a soft capsule formulation comprising BHB acid. The formulation is shown in the Table below.

| | | |
|---|---|---|
| Component name | Mass (kg) | Mass percentage (%) |

Filling material:

| | | |
|---|---|---|
| BHB acid | 18.00 | 58.06 |
| Glycerin | 1.00 | 3.23 |
| Polyethylene glycol 400 | 1.00 | 3.23 |

Outer shell:

| | | |
|---|---|---|
| Plant-based material | 6.05 | 19.52 |
| Glycerin | 2.75 | 8.87 |
| Water | 2.20 | 7.09 |

The formulation was prepared as follows:

### Step 1: Fill Material Preparation

18 kg BHB acid, 1 kg glycerol and 1 kg polyethylene glycol 400 were mixed and heated to 50° C. Then the mixture was stirred at 15000 rpm for 8 min under high shear conditions until homogenous mixture was obtained.

### Step 2: Molten Gel Preparation

In a separate container, 6.05 kg Plant-based material (i.e., a mixture of carrageenan and starches), 2.75 kg glycerin, and 6.66 kg water were mixed together, and stirred at 90° C. for 4 h until a homogenous molten gel was formed. Then, the molten gel was de-aerated under vacuum, and filtered using an 80-mesh screen, and then stored in a storage tank at constant temperature of 90° C. until used for encapsulation. During the gel manufacturing process, the ambient temperature is 20° C. and the relative humidity is 45%.

### Step 3: Encapsulation

The soft capsule is prepared by encapsulating fill material in a gel shell using a rotary die encapsulation process. In the encapsulation process, two gel ribbons are manufactured and pass between a twin set of rotating dies. As the ribbons meet, the filling formulation to be encapsulated is precisely injected between them. The capsule halves are sealed and ejected by the continuous rotation of the dies. During the encapsulation process, the ambient temperature is 20° C. and the relative humidity is 45%. The content weight is 1000 mg.

### Step 4: Drying

An intelligent soft capsule tumbler dryer was used for shaping, drying and polishing. During the process, the ambient temperature is 20° C. and the relative humidity is 45%. After drying, the moisture content of the shell is 7.09%.

### Step 5: Quality Inspection

After drying, the soft capsules were placed on the lamp inspection platform for quality inspection. During the process, the ambient temperature is 20° C. and the relative humidity is 45%.

### Step 6: Packaging

During the process, the ambient temperature is 20° C. and the relative humidity is 30%. Qualified soft capsules were packed.

### Step 7: Storage

During the process, the ambient temperature is 20° C. and the relative humidity is 45%. The finished product of BHB soft capsules were packed and stored. The soft capsule is spherical, the weight of filling material (content weight) is 1000 mg, and the total weight of the soft capsule is about 1550 mg.

### Example 6

This Example discloses a soft capsule formulation comprising BHB ester. The formulation is shown in the Table below.

| | | |
|---|---|---|
| Component name | Mass (kg) | Mass percentage (%) |

Filling material:

| | | |
|---|---|---|
| BHB ester | 30.00 | 68.18 |

Outer shell:

| | | |
|---|---|---|
| Plant-based material | 8.40 | 19.09 |
| Sorbitol | 3.36 | 7.64 |
| Water | 2.24 | 5.09 |

The formulation was prepared as follows:

### Step 1: Fill Material Preparation

30 kg of BHB ester, being a liquid, was heated to 45° C. Then the liquid was stirred at 10000 rpm for 10 min under high shear conditions.

### Step 2: Molten Gel Preparation

In a separate container, 8.40 kg Plant-based material (i.e., a mixture of locust bean gums and starches), 3.36 kg sorbitol, and 10.08 kg water were mixed together, and stirred at 100° C. for 3 h until a homogenous molten gel was formed. Then, the molten gel was de-aerated under vacuum, and filtered using an 80-mesh screen, and then stored in storage tank at constant temperature of 90° C. until used for encapsulation. During the gel manufacturing process, the ambient temperature is 20° C. and the relative humidity is 45%.

### Step 3: Encapsulation

The soft capsule is prepared by encapsulating fill material in a gel shell using a rotary die encapsulation process. In the encapsulation process, two gel ribbons are manufactured and pass between a twin set of rotating dies. As the ribbons meet, the filling formulation to be encapsulated is precisely injected between them. The capsule halves are sealed and ejected by the continuous rotation of the dies. During the encapsulation process, the ambient temperature is 20° C. and the relative humidity is 45%. The content weight is 2000 mg.

### Step 4: Drying

An intelligent soft capsule tumbler dryer was used for shaping, drying and polishing. During the process, the ambient temperature is 20° C. and the relative humidity is 30%. After drying, the moisture content of the shell is 5.09%.

### Step 5: Quality Inspection

After drying, the soft capsules were placed on the lamp inspection platform for quality inspection. During the process, the ambient temperature is 20° C. and the relative humidity is 45%.

### Step 6: Packaging

During the process, the ambient temperature is 20° C. and the relative humidity is 30%. Qualified soft capsules were packed.

### Step 7: Storage

During the process, the ambient temperature is 20° C. and the relative humidity is 45%. The finished product of BHB soft capsules were packed and stored. The soft capsule is spherical, the weight of filling material (content weight) is 2000 mg, and the total weight of soft capsule is about 2950 mg.

### Example 7

This Example discloses a soft capsule formulation comprising BHB calcium magnesium salt. The formulation is shown in the Table below.

| | | |
|---|---|---|
| Component name | Mass (kg) | Mass percentage (%) |

Filling material:

| | | |
|---|---|---|
| BHB calcium magnesium | 45.00 | 56.25 |
| salt | | |
| Polyethylene glycol 400 | 4 | 5.00 |
| Sorbitol | 1 | 1.25 |

Outer shell:

| | | |
|---|---|---|
| Plant-based material | 18.00 | 22.50 |
| Polyethylene glycol 400 | 6.60 | 8.25 |
| Water | 5.40 | 6.75 |

The formulation was prepared as follows:

### Step 1: Fill Material Preparation

45 kg BHB calcium magnesium salt, 4 kg polyethylene glycol 400 and 1 kg sorbitol were mixed and heated to 45° C. Then the mixture was stirred at 12000 rpm for 8 min under high shear conditions until homogenous mixture was obtained.

### Step 2: Molten Gel Preparation

In a separate container, 18.00 kg of Plant-based material (i.e., a mixture of sodium alginates and starches), 6.60 kg polyethylene glycol 400, and 21.60 kg water were mixed together, and stirred at 95° C. for 3.5 h until a homogenous molten gel was formed. Then, the molten gel was de-aerated under vacuum, and filtered using an 80-mesh screen, and then stored in a storage tank at a constant temperature of 95° C. until used for encapsulation. During the gel manufacturing process, the ambient temperature is 20° C. and the relative humidity is 45%.

### Step 3: Encapsulation

The soft capsule is prepared by encapsulating fill material in a gel shell using a rotary die encapsulation process. In the encapsulation process, two gel ribbons are manufactured and pass between a twin set of rotating dies. As the ribbons meet, the filling formulation to be encapsulated is precisely injected between them. The capsule halves are sealed and ejected by the continuous rotation of the dies. During the encapsulation process, the ambient temperature is 20° C. and the relative humidity is 45%. The content weight is 1000 mg.

### Step 4: Drying

An intelligent soft capsule tumbler dryer was used for shaping, drying and polishing. The ambient temperature is 20° C. and the relative humidity is 30%. After drying, the moisture content of the shell is 6.75%.

### Step 5: Quality Inspection

After drying, the soft capsules were placed on the lamp inspection platform for quality inspection. During the process, the ambient temperature is 20° C. and the relative humidity is 45%.

### Step 6: Packaging

During the process, the ambient temperature is 20° C. and the relative humidity is 30%. Qualified soft capsules were packed.

### Step 7: Storage

During the process, the ambient temperature is 20° C. and the relative humidity is 45%. The finished product of BHB soft capsules were packed and stored. The soft capsule is spherical, the weight of filling material (content weight) is 1000 mg, and the total weight of soft capsule is about 1600 mg.

### Example 8

This Example discloses a soft capsule formulation comprising BHB liposome. The formulation is shown in the Table below.

| | | |
|---|---|---|
| Component name | Mass (kg) | Mass percentage (%) |

Filling material:

| | | |
|---|---|---|
| BHB liposome | 50.00 | 66.67 |

Outer shell:

| | | |
|---|---|---|
| Plant-based material | 15.00 | 20.00 |
| Glycerin | 6.00 | 8.00 |
| Water | 4.00 | 5.33 |

The formulation was prepared as follows:

### Step 1: Fill Material Preparation

50 kg of BHB liposome, being a liquid, was heated to 50° C. Then the liquid was stirred at 24000 rpm for 5 min under high shear conditions.

### Step 2: Molten Gel Preparation

In a separate container, 15.00 kg of Plant-based material (a mixture of carrageenan and starches), 6.00 kg of glycerin, and 17.10 kg of water were mixed together, and stirred at 95° C. for 3 h until a homogenous molten gel was formed. Then, the molten gel was de-aerated under vacuum, and filtered using a 100-mesh screen, and then stored in a storage tank at constant temperature of 95° C. until used for encapsulation. During the gel manufacturing process, the ambient temperature is 20° C. and the relative humidity is 45%.

### Step 3: Encapsulation

The soft capsule is prepared by encapsulating fill material in a gel shell using a rotary die encapsulation process. In the encapsulation process, two gel ribbons are manufactured and pass between a twin set of rotating dies. As the ribbons meet, the filling formulation to be encapsulated is precisely injected between them. The capsule halves are sealed and ejected by the continuous rotation of the dies. During the encapsulation process, the ambient temperature is 20° C. and the relative humidity is 45%. The content weight is 1000 mg.

### Step 4: Drying

An intelligent soft capsule tumbler dryer was used for shaping, drying and polishing. During the process, the ambient temperature is 20° C. and the relative humidity is 30%. After drying, the moisture content of the shell is 5.33%.

### Step 5: Quality Inspection

After drying, the soft capsules were placed on the lamp inspection platform for quality inspection. During the process, the ambient temperature is 20° C. and the relative humidity is 45%.

### Step 6: Packaging

During the process, the ambient temperature is 20° C. and the relative humidity is 30%. Qualified soft capsules were packed.

### Step 7: Storage

During the process, the ambient temperature is 20° C. and the relative humidity is 45%. The finished product of BHB soft capsules were packed and stored. The soft capsule is spherical, the weight of filling material (content weight) is 1000 mg, and the total weight of the soft capsule is about 1500 mg.

### Example 9 Evaluation of Taste-Masking Effects of Soft Capsule Formulations

In this study, the inventor has investigated the capabilities of the soft capsule formulations in masking the sour taste of BHB acid and BHB liposome, the astringent taste of BHB salt, and/or the odor taste of BHB ester.

### Evaluation of the Taste-Masking Effects of Soft Capsule Formulations on the Sour Taste of BHB Acid and BHB Liposome

Ten healthy college students aged 22 to 28 were enrolled to evaluate the taste-masking effects of the soft capsule formulations of BHB acid soft capsules (Example 1) and BHB liposome soft capsules (Example 8).

The evaluation was conducted by comparing the taste of the soft capsules to the sour taste of BHB acid (the filling material of example 1, named as Comparative Example 1) and BHB liposome (the filling material of example 8, named as comparative example 8).

During evaluation, the enrolled participants rinsed their mouth after tasting each sample. And there was at least 15-minute interval of time between tasting each sample. The degree of the sour taste is set as three levels: obvious sour, slight sour, and no sour.

Results showed that all participants rated that example 1 had no sour taste in mouth while comparative example 1 had obvious sour taste, and example 8 had no sour taste in mouth while comparative example 8 had obvious/or slight sour taste (see Table 1). In conclusion, the soft capsules have capabilities to mask the sour taste of BHB acid and BHB liposome.

TABLE 1
Taste-masking effects of soft capsule formulations on sour taste

| | | Comparative | | Comparative |
|---|---|---|---|---|
| Participant | Example 1 | example 1 | Example 8 | example 8 |
| 1 | No sour | Obvious sour | No sour | Obvious sour |
| 2 | No sour | Obvious sour | No sour | Obvious sour |
| 3 | No sour | Obvious sour | No sour | Obvious sour |
| 4 | No sour | Obvious sour | No sour | Obvious sour |
| 5 | No sour | Obvious sour | No sour | Slight sour |
| 6 | No sour | Obvious sour | No sour | Obvious sour |
| 7 | No sour | Obvious sour | No sour | Obvious sour |
| 8 | No sour | Obvious sour | No sour | Obvious sour |
| 9 | No sour | Obvious sour | No sour | Obvious sour |
| 10 | No sour | Obvious sour | No sour | Obvious sour |

### Evaluation of the Taste-Masking Effects of Soft Capsule Formulations on the Odor Taste of BHB Ester

Ten healthy college students aged 22 to 28 were enrolled to evaluate the taste-masking effects of the soft capsule formulation of BHB ester soft capsules (Example 2 and Example 6).

The evaluation is conducted by comparing the taste of the soft capsules to the odor taste of BHB ester (the filling material of example 2, named as comparative example 2).

During evaluation, the enrolled participants rinsed their mouth after tasting each sample. And there was at least 15-minute interval of time between tasting each sample. The degree of the odor taste is set as three levels: obvious odor, slight odor, and no odor.

Results showed that all participants rated that example 2 and example 6 had no odor taste in mouth while comparative example 2 had obvious odor (see Table 2). In conclusion, the soft capsules have capabilities to mask the odor taste of BHB ester.

TABLE 2
Taste-masking effects of soft capsule formulations on odor taste

| | | | Comparative |
|---|---|---|---|
| Participant | Example 2 | Example 6 | example 2 |
| 1 | No odor | No odor | Obvious odor |
| 2 | No odor | No odor | Obvious odor |
| 3 | No odor | No odor | Obvious odor |
| 4 | No odor | No odor | Obvious odor |
| 5 | No odor | No odor | Obvious odor |
| 6 | No odor | No odor | Obvious odor |
| 7 | No odor | No odor | Obvious odor |
| 8 | No odor | No odor | Obvious odor |
| 9 | No odor | No odor | Obvious odor |
| 10 | No odor | No odor | Obvious odor |

### Evaluation of Taste-Masking Effects of Soft Capsule Formulations on the Astringent Taste of BHB Salt

Ten healthy college students aged 22 to 28 were enrolled to evaluate the taste-masking effects of the soft capsule formulations of BHB sodium salt soft capsule (Example 3) BHB calcium magnesium salt soft capsule (Example 7).

The evaluation is conducted by comparing the taste of the soft capsules to the astringent taste of BHB sodium salt (the filling material of example 3, named as comparative example 3), and BHB calcium magnesium salt (the filling material of example 7, named as comparative example 7).

During evaluation, the enrolled participants rinsed their mouth after tasting each sample. And there was at least 15-minute interval of time between tasting each sample. The degree of the astringent taste is set as three levels: obvious astringency, slightly astringency, no astringency.

Results showed that all participants rated that example 3 had no astringency taste in mouth while comparative example 3 had slight or obvious astringency, example 7 had no astringency taste in mouth while comparative example 7 had slight or obvious astringency (see Table 3). In conclusion, the soft capsules have the capabilities to mask the astringent taste of BHB salt.

TABLE 3
Taste-masking effects of soft capsule formulations on astringent taste

| | | Comparative | | Comparative |
|---|---|---|---|---|
| Participant | Example 3 | example 3 | Example 7 | example 7 |
| 1 | No astringency | Slight astringency | No astringency | Slight astringency |
| 2 | No astringency | Obvious astringency | No astringency | Obvious astringency |
| 3 | No astringency | Obvious astringency | No astringency | Obvious astringency |
| 4 | No astringency | Obvious astringency | No astringency | Obvious astringency |
| 5 | No astringency | Slight astringency | No astringency | Slight astringency |
| 6 | No astringency | Obvious astringency | No astringency | Obvious astringency |
| 7 | No astringency | Obvious astringency | No astringency | Obvious astringency |
| 8 | No astringency | Obvious astringency | No astringency | Obvious astringency |
| 9 | No astringency | Obvious astringency | No astringency | Obvious astringency |
| 10 | No astringency | Obvious astringency | No astringency | Obvious astringency |

Although specific embodiments and examples of this invention have been illustrated herein, it will be appreciated by those skilled in the art that any modifications and variations can be made without departing from the spirit of the invention. The examples and illustrations above are not intended to limit the scope of this invention. Any combination of embodiments of this invention, along with any obvious their extension or analogs, are within the scope of this invention. Further, it is intended that this invention encompass any arrangement, which is calculated to achieve that same purpose, and all such variations and modifications as fall within the scope of the appended claims.

All the features disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example of a generic series of equivalent or similar features.

What is claimed is:

1. A soft capsule comprising an outer shell and a fill material within the outer shell, wherein the outer shell comprises a gelatin or a plant-based material; the fill material comprises an active ingredient which is β-hydroxybutyric acid (BHB), a pharmaceutically acceptable salt, ester, liposome, or a mixture thereof.

2. The soft capsule of claim 1, wherein the outer shell further comprises a humectant and water, the humectant is selected from polyethylene glycol 200, polyethylene glycol 400, polyethylene glycol 600, polyethylene glycol 800, polyethylene glycol 1000, polyethylene glycol 1500, polyethylene glycol 2000, glycerin, sorbitol, polydextrose, maltitol, or a mixture thereof.

3. The soft capsule of claim 2, wherein the humectant is selected from glycerin, sorbitol or polyethylene glycol 400.

4. The soft capsule of claim 2, wherein the active ingredient has a mass percentage ranging from 20% to 80% of the total weight of the capsule; the gelatin or plant-based material has a mass percentage ranging from 10% to 40% of the total weight of the capsule; the humectant has a mass percentage ranging from 2% to 20% of the total weight of the capsule; the water has a mass percentage ranging from 2% to 20% of the total weight of the capsule.

5. The soft capsule of claim 1, wherein the gelatin has a bloom strength of 100-300; the plant-based material is starch or colloid.

6. The soft capsule of claim 5, wherein the starch is selected from oxidized starch, starch dispersion, starch derivatives, starch hydrolysate, wet heat-treated starch, acid-treated starch, or a mixture thereof; the colloid is selected from carrageenan, mannan gum, xanthan gum, alginate, or a mixture thereof.

7. The soft capsule of claim 1, wherein the pharmaceutically acceptable salt, ester is selected from BHB potassium salt, BHB sodium salt, BHB calcium salt, BHB magnesium salt, BHB calcium magnesium salt, BHB triglyceride.

8. The soft capsule of claim 7, wherein the pharmaceutically acceptable salt, ester is selected from BHB sodium salt, BHB calcium magnesium salt, BHB triglyceride.

9. The soft capsule of claim 1, wherein the fill material further comprises a suspending agent, the suspending agent is selected from polyethylene glycol 200, polyethylene glycol 400, polyethylene glycol 600, polyethylene glycol 800, polyethylene glycol 1000, polyethylene glycol 1500, polyethylene glycol 2000, glycerin, sorbitol, polydextrose, maltitol and a mixture thereof.

10. The soft capsule of claim 9, wherein the suspending agent is glycerin, polyethylene glycol 400 or sorbitol.

11. The soft capsule of claim 9, wherein the suspending agent has a mass percentage ranging from 3% to 45% of the total weight of the capsule.

12. The soft capsule of claim 1, wherein the fill material is in the form of solution, suspension, or paste.

13. The soft capsule of claim 12, wherein the fill material is in the form of suspension.

14. The soft capsule of claim 1, wherein the weight of the fill material ranges from 80 to 2500 mg, and total weight of the soft capsule ranges from 120 to 3750 mg.

15. A method for preparing a soft capsule of claim 1, comprising the steps of (a) preparing β-hydroxybutyric acid (BHB), or a pharmaceutically acceptable salt, ester, liposome or mixture thereof, to form a fill composition; (b) preparing and formulating an outer shell using gelatin or a plant-based material; and (c) encapsulating the fill material into the outer shell.

16. The method of claim 15, further comprising a step of shaping, drying, quality inspection, packaging, or storage.

17. The method of claim 15, wherein step (a) comprises mixing the BHB or pharmaceutically acceptable salt with a suspending agent together, heating the mixture to a temperature of 35-60° C. and stirring, to form a homogenous mixture; or heating the pharmaceutically acceptable ester or liposome to a temperature of 35-60° C. and stirring.

18. The method of claim 15, wherein step (b) comprises mixing the gelatin or plant-based material with a humectant and water together, stirring and heated to 50-100° C., de-aerating and filtering, to formulate an outer shell liquid.
